(19) United States
(12) Patent Application Publication
(10) Pub. No.: US 2026/0137555 A1
(43) Pub. Date: May 21, 2026

(54) Infrared light therapy with cold-water immersion

(71) Applicant: Nick Reed, Sheridan, WY (US)

(72) Inventor: Nick Reed, Sheridan, WY (US)

(21) Appl. No.: 19/362,393

(22) Filed: Oct. 18, 2025

Related U.S. Application Data
(63) Continuation-in-part of application No. 18/955,946, filed on Nov. 21, 2024.

(51) Int. Cl. A61F 7/00; A61N 5/06

(52) U.S. Cl. CPC A61F 7/0085; A61N 5/0616; A61F 2007/0003; A61F 2007/0093; A61N 2005/0626; A61N 2005/0659; A61N 2005/0663; A61N 2005/0667

(57) ABSTRACT

A system for performing cold immersion with infrared light therapy, comprising: a reservoir or plunge chamber configured to contain water and submerge target tissue below a waterline; a set of near-infrared emitters positioned below the waterline and oriented toward the target tissue; a water-temperature sensor configured to measure and relay real-time temperature data to a controller; and a control system configured to: deliver light at 800-880 nm with an average irradiance of 60-160 mW/cm² through 15-40 mm of water for 20-180 seconds; suppress spectral radiant power greater than 1400 nm by virtue of the water column; maintain epidermal temperature below 32° C. without direct skin sensing, using the measured water temperature and a pre-calibrated irradiance-time model; and limit session duration via a programmable timer to ensure therapeutic safety and consistency.

### BACKGROUND OF THE INVENTION

### Field of Invention

The present invention relates generally to devices and systems for cold therapy, particularly to portable, compact apparatuses designed for facial and body immersion in cold water. More specifically, this invention pertains to a facial cold plunge device with integrated light therapy capability for therapeutic, cosmetic, and wellness applications. It finds utility in the health, wellness, and beauty industries, providing users with improved access to the physiological and aesthetic benefits.

### BRIEF SUMMARY OF THE INVENTION

The present invention provides a system for performing cold immersion with infrared light therapy, comprising: a reservoir or plunge chamber configured to contain water and submerge target tissue below a waterline; a set of near-infrared emitters positioned below the waterline and oriented toward the target tissue; a water-temperature sensor configured to measure and relay real-time temperature data to a controller; and a control system configured to: deliver light at 800-880 nm with an average irradiance of 60-160 mW/cm² through 15-40 mm of water for 20-180 seconds; suppress spectral radiant power greater than 1400 nm by virtue of the water column; maintain epidermal temperature below 32° C. without direct skin sensing, using the measured water temperature and a pre-calibrated irradiance-time model; and limit session duration via a programmable timer to ensure therapeutic safety and consistency.

The invention is particularly applicable in health and wellness industries, providing benefits such as stress reduction, vagus nerve stimulation, inflammation reduction, and improved skin tone and texture.

### BRIEF DESCRIPTION OF THE FIGURES

FIG. 1: illustrates the key components of the facial cold plunge device, including the adjustable thermostat (102), filter (106), IR light (108), removable mouthpiece for cleaning (110), lowering attached breathing mechanism (112), and GFCI outlet (114), highlighting its functional and safety features.

FIG. 2: The operational flow of the facial cold plunge device involves preparing the reservoir, activating and configuring the device, engaging its chilling and filtration systems, positioning the breathing apparatus, safely immersing the face, and performing post-use maintenance to ensure continued functionality.

### DETAILED DESCRIPTION

The present invention further includes an advanced variation integrating infrared (IR) and near-infrared (NIR) light therapy with cold water immersion, providing combined therapeutic benefits for skin health, stress reduction, and enhanced healing. This variation utilizes the water's scattering and heat-conductive properties to optimize the distribution and safety of infrared light while maintaining the cooling benefits of the device.

In this configuration, the ergonomic bowl-shaped reservoir is equipped with infrared and near-infrared light sources embedded in a light pad positioned at the base of the bowl. The light pad emits IR and NIR light through the water, with the water serving as a medium to scatter the light evenly across the submerged face. This design ensures uniform exposure and prevents the light from escaping beyond the waterline. The light pad is backed by a heat-resistant insulating layer to prevent overheating of the components and condensation on the cold surface of the bowl. The light intensity is carefully calibrated to leverage the water's thermal conductivity, allowing for the safe use of higher intensity levels than conventional air-based systems.

The infrared system is operably connected to a power source equipped with a timer to limit session durations, ensuring user safety while maintaining therapeutic efficacy. The user activates the lights via a power button located on the device. Alternatively, an optional snorkel trigger mechanism may be included, which automatically activates the lights when the snorkel is submerged and deactivates them when the snorkel returns to its default position.

The design also incorporates specific features to protect the eyes from direct infrared exposure. A scuba mask-like seal surrounds the eyes, ergonomically fitting the user's face to block light while leaving the eye area exposed to cold water for puffiness reduction. The combination of cold water and infrared exposure reduces the traditional 20-30 minute therapy duration to just a few minutes, enhancing convenience while maintaining effectiveness.

This enhanced variation is particularly beneficial for users with sensitive skin who may experience dryness or redness from traditional infrared therapy. The cold water circulation, maintained by the integrated chilling and filtration system, ensures that any localized heating effects from infrared exposure are mitigated, preserving the skin's health while amplifying therapeutic benefits.

Additionally, the invention includes a larger variation designed for integration into a full-body cold plunge system. Infrared and near-infrared light pads are installed along the side walls of the plunge tub, with light shining through perforations in the liner. This setup utilizes the cooling effects of the water to safely accommodate higher light intensity levels, enabling users to experience enhanced benefits of simultaneous cold and infrared therapy in reduced session times. The system includes a GFCI-protected power source and a timer to ensure safety and optimal use.

These innovations make the infrared-integrated facial cold plunge device and its full-body variation uniquely positioned to deliver dual therapeutic benefits efficiently, addressing issues of skin sensitivity, overheating, and prolonged therapy durations that are common with traditional infrared systems.

The present invention further encompasses a method for delivering photobiomodulation therapy to submerged tissue by combining near-infrared (NIR) light exposure with cold water immersion in a manner that enhances both safety and therapeutic efficacy. In one embodiment, the method comprises immersing a portion of the user's body—including but not limited to the face—in water maintained at or below 50° F. (10° C.) and simultaneously irradiating the submerged tissue with near-infrared light in the wavelength range of 800-880 nm. The light is transmitted through a defined optical path of cold water ranging from 15 to 40 mm in thickness, with an average irradiance of 60 to 160 mW/cm² measured at the tissue plane.

The water acts both as a spectral filter—suppressing wavelengths above 1400 nm associated with undesirable thermal effects—and as a thermal sink, enabling short-duration, high-intensity light exposure while maintaining epidermal temperature below 32° C. The method delivers a fluence of at least 3 to 10 J/cm² to the dermal or sub-dermal layers within 20 to 180 seconds of exposure. Safety is ensured not by direct skin-temperature sensing but by use of calibrated irradiance-time models in combination with measured water temperature, obviating the need for external thermal probes.

In embodiments where the face is submerged, the cold-water immersion triggers vagus nerve activation through the mammalian diving reflex, producing measurable autonomic responses, including a reduction in heart rate of at least 5-15% within 60-120 seconds and an increase in heart rate variability (RMSSD) of at least 10-20% within five minutes post-treatment. In addition to autonomic effects, cosmetic benefits are also observed, including reduced periorbital puffiness and improved skin tone and firmness, without erythema exceeding 5% over baseline at 15-60 minutes post-treatment.

The method further comprises a pulsed irradiation mode wherein the NIR light source is driven at a frequency of 10-100 Hz with a duty cycle of 25-50%, or in a quasi-continuous mode around 1 kHz. This pulsed mode may be optionally activated to enhance contrast therapy effects by mimicking physiological fluctuations while preserving dose delivery within established safety parameters. In some embodiments, a secondary red-light emitter operating in the 630-670 nm range may be included to deliver ≥3 J/cm² to submerged tissue in parallel with NIR exposure.

The present invention provides for a face-only cold-plunge device with pulse-only NIR therapy. One preset for NIR (40 Hz, 50% duty), one fixed run (50 s). At the face plane (through ˜1″ water) the NIR is ˜280 mW/cm² on-pulse which averages ˜140 mW/cm²; all dosing/compliance/UI use the average. A 50 s session delivers 6-8 J/cm² NIR across the accepted average range (120-160 mW/cm²). Water operates 45-55° F. (7-12.8° C.); NIR won't start above 55° F. All treatment LEDs are below the fill line; water +geometry provide optical/thermal filtering. Includes peltier model chilling to 7° C. in ˜30 min, UV sanitation, replaceable/washable cartridge filter with flow-based quality sensor+LCD alerts, overflow moat, drain pump, rim lighting, snorkel, water-seal goggles, GFCI cord; targets UL/ETL.

By leveraging the water's optical and thermal properties in combination with defined irradiance protocols and outcome targets, the method offers a significant advancement over traditional infrared therapies conducted in air. It enables shorter, safer, and more effective treatment sessions while producing both physiological and cosmetic benefits previously unattainable through prior methods.

The present invention is directed to a novel and advanced facial cold plunge device, which integrates a suite of technical features to enable prolonged and effective facial immersion in cold water for therapeutic, cosmetic, and wellness applications. The invention comprises a main housing unit fabricated from thermally insulated materials, such as high-grade thermoplastics or stainless steel, designed to maintain the thermal integrity of the internal water reservoir.

The housing contains an ergonomically contoured, bowl-shaped reservoir specifically designed to conform to the user's facial profile, enhancing comfort and immersion efficiency while minimizing water spillage. Embedded beneath the reservoir is an integrated chilling mechanism employing a miniature compressor or thermoelectric cooling system.

This system incorporates evaporative cooling coils positioned strategically to ensure uniform cooling across the reservoir, maintaining water temperatures below 50° F.

The chilling mechanism is controlled via an electronic thermostat interface, equipped with a digital display for real-time temperature readings and programmable settings, allowing the user to customize and maintain precise water temperatures for optimal therapeutic effect.

The device further incorporates a closed-loop water circulation system powered by a compact, low-energy pump housed within the base unit. This pump continuously circulates water through a filtration assembly, which includes replaceable carbon or HEPA filters to remove impurities, bacteria, and debris, ensuring the water remains clean and suitable for repeated use without requiring frequent draining or replacement.

The circulation system also incorporates insulated conduits to prevent thermal losses during operation, ensuring consistent and efficient cooling. A unique feature of the invention is the retractable breathing apparatus, which allows users to sustain facial submersion for extended durations without the need to interrupt the immersion process to breathe. This apparatus comprises a snorkel-like breathing tube constructed of medical-grade silicone, which is mounted on the housing unit adjacent to the reservoir.

The tube is mechanically retractable via a spring-based or tensioned arm mechanism, allowing it to be stowed when not in use. The breathing tube is further equipped with a two-way check valve system that regulates airflow, preventing water ingress while facilitating effortless inhalation and exhalation during submersion. The height and position of the breathing tube can be adjusted to align with the user's facial orientation, enhancing comfort and usability.

The invention's structural and functional design prioritizes portability and convenience. The housing unit is compact and lightweight, enabling the device to be easily placed in residential or commercial settings, including small apartments or high-rise buildings where space and access to full-body cold plunges may be limited. Rubberized feet or suction cups on the base provide stability during use, while a detachable power cord or optional battery-operated configuration enhances placement flexibility.

Operationally, the device is user-friendly and efficient. Users fill the reservoir with water to a designated level, activate the chilling and filtration systems, and set their desired water temperature via the thermostat. Once the water reaches the target temperature, the user submerges their face into the reservoir, with the ergonomic design ensuring a secure and comfortable fit. For extended immersions, the user employs the breathing apparatus, allowing for continuous respiration while benefiting from the therapeutic effects of cold water immersion.

The present invention provides significant advantages over existing solutions, addressing numerous limitations in traditional facial cold therapy methods. The integrated chilling system eliminates the need for external ice and the associated waiting periods for cooling, while the filtration system ensures hygienic water conditions without requiring daily maintenance or water changes. The retractable breathing mechanism offers a hands-free solution to the common challenge of holding one's breath during immersion, enabling longer, uninterrupted sessions.

Yet another aspect of the invention is the compact design, which allows for placement in diverse environments, making the invention accessible to individuals who may not have the space or resources for full-body cold plunges. Therapeutic benefits include stimulation of the vagus nerve via the mammalian diving reflex, reduction of stress and anxiety, decreased inflammation, and cosmetic improvements such as reduced puffiness, enhanced skin tone, and minimized signs of aging. The precise temperature control ensures consistent results, a significant improvement over the variability of DIY methods, where the temperature often fluctuates due to melting ice or inconsistent water chilling.

This invention introduces a groundbreaking approach to facial cold therapy, combining state-of-the-art technology with ergonomic and aesthetic design. The integration of chilling, filtration, and respiratory support systems into a single, portable unit offers unparalleled convenience and effectiveness. By providing clean, cold water at a consistent temperature with a seamless user experience, this device sets a new standard for cold therapy applications in the health, wellness, and beauty industries. Its innovative design and advanced technical features make it uniquely positioned to address the unmet needs of individuals seeking accessible, efficient, and effective cold therapy solutions.

The present invention further includes a variation designed to attach to the wall of a full-body cold plunge, enabling the integration of facial immersion therapy into an existing system. In this configuration, the device features an adaptable mounting mechanism that securely affixes the ergonomic reservoir to the rim or sidewall of a full-body plunge tub.

A submersible pump, operatively connected to the main unit, draws water from the full-body plunge tub into the facial immersion bowl and recirculates it back into the tub. This closed-loop circulation system eliminates the need for separate chilling and filtration mechanisms within the facial device itself, as the water is maintained at the desired temperature and cleanliness by the full-body plunge's primary systems.

The breathing apparatus, ergonomic reservoir, and optional UV sterilization components remain integral to this variation, ensuring users experience the same therapeutic and cosmetic benefits while leveraging existing equipment. This configuration enhances the versatility and cost-effectiveness of the invention, making it accessible to users who already utilize full-body cold plunge systems but desire the convenience and specificity of facial cold therapy.

### DETAILED DESCRIPTION OF FIGURES

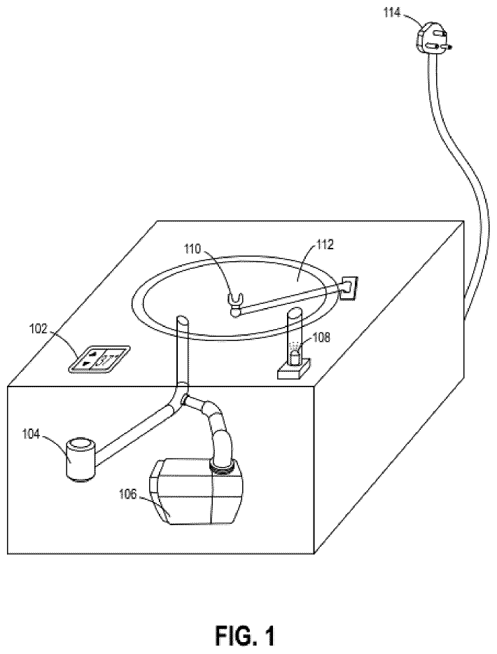

FIG. 1: illustrates the structural and functional components of the facial cold plunge device, showcasing its innovative features that facilitate effective and user-friendly operation. The following components are labeled with their respective reference numerals:
102: Adjustable Thermostat

The adjustable thermostat, integrated into the device, allows users to precisely set and maintain the desired water temperature. The thermostat includes a digital interface displaying real-time temperature readings and programmable controls for custom settings.
106: Filter

The filtration system, identified as component 106, ensures water hygiene by removing impurities, bacteria, and debris. The filter is replaceable and part of the closed-loop water circulation system, maintaining the cleanliness of the water for multiple uses.
108: UV Light

Positioned adjacent to the filtration system, the UV light sterilizes the circulating water by eliminating microbial contaminants, further enhancing water purity and safety during prolonged immersion.
110: Removable Mouthpiece for Easy Cleaning

The mouthpiece of the breathing apparatus, designated as 110, is detachable to facilitate thorough cleaning. Constructed from medical-grade silicone, it ensures hygiene and comfort during repeated usage.
112: Lowering Attached Breathing Mechanism

The attached breathing mechanism, referenced as 112, is a retractable snorkel-like tube that lowers into position for use. This component includes an adjustable height mechanism and a two-way check valve to allow seamless respiration while preventing water ingress during facial immersion.
114: GFCI Outlet

The Ground Fault Circuit Interrupter (GFCI) outlet, labeled 114, ensures electrical safety during operation, protecting the user and device from potential electrical faults, particularly in wet environments.

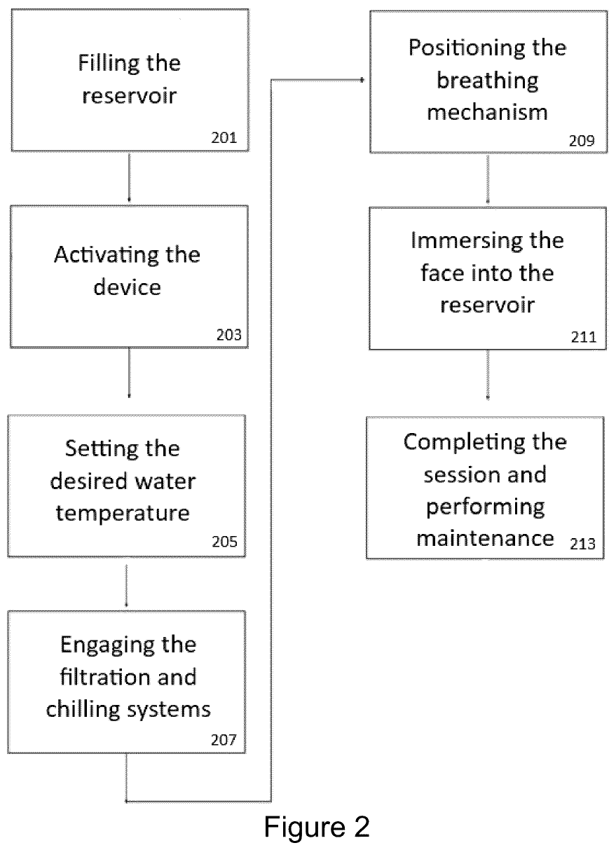

FIG. 2.201: Filling the Reservoir

The first step involves filling the ergonomically contoured reservoir with water to the indicated fill line. The reservoir is designed to accommodate an optimal volume for immersion, ensuring sufficient water depth for effective facial coverage while minimizing overflows or spillage.

FIG. 2.203: Activating the Device—After the reservoir is filled, the device is powered on by connecting it to a power source and engaging the GFCI outlet for safety. The internal systems, including the thermostat, filtration, and chilling mechanisms, initialize upon activation, preparing the device for operation.

FIG. 2.205: Setting the Desired Water Temperature—Using the adjustable thermostat, the user selects the desired water temperature. The thermostat interface displays real-time temperature readings and allows for precise adjustment to achieve optimal cooling below 50° F. This step ensures consistent temperature settings for effective cold therapy.

FIG. 2.207: Engaging the Filtration and Chilling Systems—Once the temperature is set, the water circulation begins as the pump activates, channeling water through the filtration assembly to remove impurities. Simultaneously, the chilling mechanism, utilizing a compressor or thermoelectric system, ensures the water cools uniformly to the pre-selected temperature.

FIG. 2.209: Positioning the Breathing Mechanism—The retractable breathing apparatus is adjusted for the user's facial alignment. The snorkel-like tube lowers into position via its spring-loaded mechanism, and the user aligns the mouthpiece for comfortable respiration. The two-way check valve prevents water ingress while facilitating seamless airflow.

FIG. 2.211: Immersing the Face into the Reservoir—The user submerges their face into the cold water reservoir, ensuring a snug fit against the ergonomic contours of the reservoir to minimize spillage. The breathing apparatus enables prolonged immersion without the need to interrupt for breaths, optimizing the therapeutic effects of the session.

FIG. 2.213: Completing the Session and Performing Maintenance—Upon completion, the user removes their face from the reservoir, powers off the device, and detaches the mouthpiece for cleaning. The filtration assembly is inspected, and any necessary water replacements are made, ensuring the device remains clean and operational for future use.

What is claimed is:

1) A system for performing cold immersion with infrared light therapy, comprising:
a reservoir or plunge chamber configured to contain water and submerge target tissue below a waterline;
a) a set of near-infrared emitters positioned below the waterline and oriented toward the target tissue;
b) a water-temperature sensor configured to measure and relay real-time temperature data to a controller;
c) and a control system configured to:
i) deliver light at 800-880 nm with an average irradiance of 60 -160 mW/cm² through 15-40 mm of water for 20-180 seconds;
ii) suppress spectral radiant power greater than 1400 nm by virtue of the water column;
iii) maintain epidermal temperature below 32° C. without direct skin sensing, using the measured water temperature and a pre-calibrated irradiance-time model; and
iv) limit session duration via a programmable timer to ensure therapeutic safety and consistency.

2) The system of claim 1, wherein the emitters are recessed or baffled such that direct emission above the waterline at a point 10 cm above the surface does not exceed 1 mW/cm².

3) The system of claim 1, wherein the controller is configured to automatically activate a pulsed mode between 10-100 Hz to enhance contrast-therapy effects while maintaining the total optical dose and thermal limits defined in claim 11.

4) The device of claim 1, wherein the reservoir is contoured to form a watertight seal with the user's face to minimize water spillage during immersion.

5) The device of claim 1, wherein the chilling mechanism is operable to cool water within the reservoir to between 32° F. and 50° F. (0° C.-10° C.) while maintaining uniform temperature distribution across the reservoir.

6) The device of claim 1, wherein the filtration assembly comprises a carbon, HEPA, or ultraviolet (UV) sterilization filter to remove bacteria and impurities.

7) The device of claim 1, wherein the breathing apparatus is retractable by a spring-loaded or tensioned arm mechanism and is detachable for cleaning.

8) The device of claim 1, wherein the housing unit further comprises a Ground Fault Circuit Interrupter (GFCI) power outlet to ensure electrical safety in wet environments.

9) The device of claim 1, further comprising an infrared and near-infrared (NIR) light pad integrated beneath the waterline of the reservoir, the light pad configured to emit wavelengths between 800-880 nm through the water for photobiomodulation therapy.

10) The device of claim 7, wherein the infrared light pad is coupled to a timer-controlled power circuit and heat-insulating backing to prevent component overheating and condensation.

11) The device of claim 7, wherein the infrared light pad is operably connected to a snorkel trigger mechanism configured to activate light emission upon submersion of the breathing apparatus.

12) The device of claim 1, further comprising an attachment mechanism adapted to mount the reservoir to a wall or rim of a full-body cold plunge tub, wherein a submersible pump draws water from the tub into the facial reservoir and recirculates it back to the tub.

13) A method of delivering therapeutic cold immersion with infrared light therapy, comprising:
a) immersing at least a portion of a user's body including the face in water maintained at or below 50° F. (10° C.);
b) irradiating the submerged tissue with near-infrared light at wavelengths between 800-880 nm at an average irradiance of 60-160 mW/cm² measured through a water path of 15-40 mm for a duration of 20-180 seconds;
c) wherein the water acts as an optical filter attenuating wavelengths greater than 1400 nm and as a thermal sink maintaining the epidermal temperature below 32° C.;
d) and wherein the irradiation delivers at least 3-10 J/cm² to the dermal or sub-dermal tissue, producing:
i) autonomic modulation defined by a heart-rate reduction of at least 5-15% within 60-120 seconds and/or a heart-rate variability (RMSSD) increase of at least 10-20% within five minutes post-treatment; and
ii) cosmetic improvement defined by periorbital puffiness reduction and improved skin tone or firmness within 15-60 minutes without erythema greater than 5% over baseline.

14) The method of claim 13, wherein the water temperature is maintained between 45-50° F. (7-10° C.) and the optical path thickness is between 20-30 mm±5 mm.

15) The method of claim 13, wherein the irradiation is pulsed at a frequency of 10-100 Hz with a duty cycle of 25-50%, or in quasi-continuous mode at approximately 1 kHz, to induce a rapid thermal-contrast effect.

16) The method of claim 13, further comprising a red-light emission component at 630-670 nm delivering at least 3 J/cm² to the submerged tissue concurrently with near-infrared exposure.

17) The method of claim 13, wherein the system reduces pulse duty cycle automatically when the water temperature exceeds 13° C. or when a pre-calibrated irradiance-time model predicts the epidermal temperature would exceed 32° C.

18) The method of claim 13, wherein irradiation uniformity at the facial plane is at least 80% within ±20% of the center with hotspot intensity not exceeding twice the mean irradiance.

19) The method of claim 13, wherein the cold-water immersion concurrently triggers vagus-nerve activation via the mammalian diving reflex, producing measurable autonomic regulation and enhanced heart-rate variability.
